(19) United States
(12) Patent Application Publication
(10) Pub. No.: US 2014/0051930 A1
(43) Pub. Date: Feb. 20, 2014

(54) SURGICAL RETRACTOR AND METHOD OF USE

(71) Applicant: Thomas E. Drochner, Memphis, TN (US)

(72) Inventor: Thomas E. Drochner, Memphis, TN (US)

(73) Assignee: Warsaw Orthopedic, Inc., Warsaw, IN (US)

(21) Appl. No.: 13/586,610

(22) Filed: Aug. 15, 2012

(51) Int. Cl. A61B 1/32

(52) U.S. Cl. 600/203; 600/206

(57) ABSTRACT

A retractor includes a base. A blade is connected to the base and extends between a first end and a second end. The blade includes at least one cavity configured to facilitate movement of the second end relative to the blade. A first member is disposed with the first end and connected to the second end. The first member is engageable to dispose the second end between a first linear orientation and a second non-linear orientation. Systems and methods are disclosed.

### TECHNICAL FIELD

The present disclosure generally relates to medical devices for the treatment of musculoskeletal disorders, and more particularly to a surgical system and method for accessing a surgical site to facilitate treatment.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, discectomy, laminectomy and implantable prosthetics. Surgical retractors may be employed during a surgical treatment to provide access and visualization of a surgical site. Such retractors space apart and support tissue and/or other anatomical structures to expose anatomical structures adjacent the surgical site and/or provide a surgical pathway to the surgical site. This disclosure describes an improvement over these prior art technologies.

### SUMMARY

Accordingly, a surgical system and method are provided for accessing a surgical site, which may include, for example, a portion of a spine to facilitate treatment thereof. It is contemplated that the surgical system and method may be employed for exposing and providing a surgical pathway to a surgical site.

In one embodiment, in accordance with the principles of the present disclosure, a surgical retractor is provided. The retractor includes a base. A blade is connected to the base and extends between a first end and a second end. The blade includes at least one cavity configured to facilitate movement of the second end relative to the blade. A first member is disposed with the first end and connected to the second end. The first member is engageable to dispose the second end between a first linear orientation and a second non-linear orientation.

### BRIEF DESCRIPTION OF THE DRAWINGS

The present disclosure will become more readily apparent from the specific description accompanied by the following drawings, in which:

FIG. 1 is a side view of one embodiment of components of a system in accordance with the principles of the present disclosure;

FIG. 2 is a side view of the components shown in FIG. 1;

FIG. 3 is a side view of one embodiment of components of a system in accordance with the principles of the present disclosure;

FIG. 4 is a side view of the components shown in FIG. 3;

FIG. 5 is a side view of the components shown in FIG. 3 disposed with tissue;

FIG. 6 is a side view of one embodiment of components of a system in accordance with the principles of the present disclosure; and

FIG. 7 is a side view of the components shown in FIG. 6.

Like reference numerals indicate similar parts throughout the figures.

### DETAILED DESCRIPTION

The exemplary embodiments of the surgical system and related methods of use disclosed are discussed in terms of medical devices for the treatment of musculoskeletal disorders and more particularly, in terms of a surgical system for accessing a spine to facilitate treatment thereof and a method for treating a spine. One or all of the system components may be reusable or disposable. The surgical system may be configured as a kit with multiple sized and configured components.

In one embodiment, the system includes a surgical retractor having a relatively thin and/or low profile blade. It is contemplated that the low profile blade prevents the blade from catching or snagging undesired anatomy. It is contemplated that the low profile blade allows a practitioner to insert the blade without unnecessarily disrupting tissue and/or anatomical structures.

In one embodiment, the surgical retractor includes a long thin blade having a slight curve to ensure the desired anatomy is trapped behind the retractor and will not slip underneath. In one embodiment, the surgical retractor is employed with a method such that the blade is inserted in a straight or flat configuration. For example, upon insertion of the blade, a tip of the blade can be articulated to form a curved configuration. This configuration allows the blade to be inserted having a low profile and formed to a desired shape to retract tissue and/or anatomical structures. It is contemplated that the curved blade holds back desired anatomical structures from a working channel at a surgical site.

In one embodiment, the articulating blade is initially in a straight configuration. In one embodiment, a rod or cable wire is disposed from the top of the blade to the bottom such that the rod or cable wire is captured at the bottom of the blade. For example, rotation of a collar pulls the rod or cable wire taught, which pulls the blade into a curved configuration. It is envisioned that in the curved configuration, cutouts in the blade are pulled together.

In one embodiment, the surgical retractor includes cut outs disposed through part of the blade, which when merged together, form a predetermined shaped blade. In one embodiment, a sheath covers the cut out sections to prevent the cut outs from pinching anatomy. In one embodiment, the blade can include a built-in or added-on sheath. It is envisioned that the sheath prevents pinching of tissue when the cutout sections are pulled together. It is contemplated that a sheath for the blade tip protects any anatomical structures from being caught or pinched in cut-outs of the blade as sections of the blade are pulled together. It is envisioned that the sheath could be built into the blade, and/or added on or disposable. In one embodiment, the surgical retractor includes cut outs disposed through a whole blade width forming separate sections that are pulled together in a configuration to make a curved configuration.

In one embodiment, the surgical retractor includes a threaded collar mechanism to articulate the blade between positions. It is contemplated that a trigger mechanism or spool and cable mechanism can also be utilized. In one embodiment, a trigger mechanism pulls a cable/rod to actuate the blade tip. It is envisioned that the trigger can be built into the handle used to hold the blade. It is envisioned that the spooling of the cable mechanism includes a trigger, which uses a rotation mechanism to pull the cable and spin around a spool to induce the articulation of the blade tip.

In one embodiment, the retractor blade has cutouts, which actuate to curve the tip of the blade. In one embodiment, the cutouts extend part way through the blade. In one embodiment, the cutouts extend completely through the blade such that there are separate sections pulled together to make a curved configuration.

In one embodiment, a rotating collar actuates articulation of the tip. In one embodiment, the collar is threaded and at least part of a first member, described herein, is threaded. It is contemplated that the first member can include solid rod/braided cable, solid rod/Nitinol wire, cable, and/or Nitinol wire. In one embodiment, the first member can have several branches.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed surgical system and methods may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, blood vessels, ligaments, tendons, cartilage and/or bone unless specifically referred to otherwise.

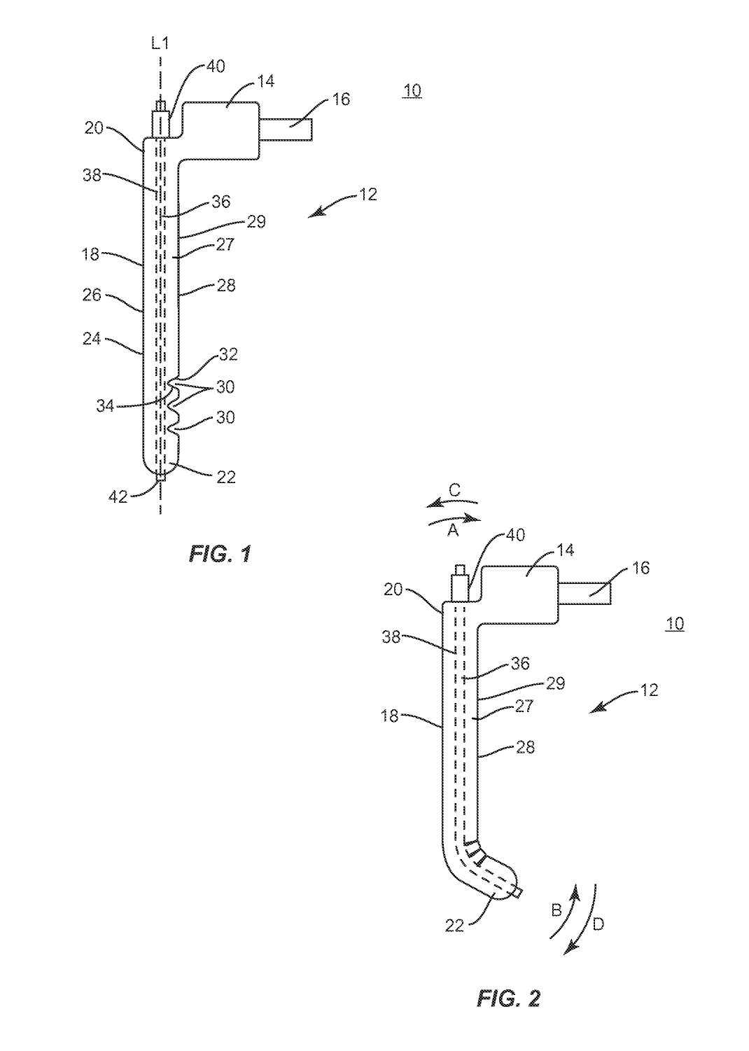

The following discussion includes a description of a surgical system and related methods of employing the surgical system in accordance with the principles of the present disclosure. Alternate embodiments are also disclosed. Reference will now be made in detail to the exemplary embodiments of the present disclosure, which are illustrated in the accompanying figures. Turning now to FIGS. 1-5, there is illustrated components of a surgical system 10 including a retractor for accessing a spine to facilitate treatment thereof in accordance with the principles of the present disclosure.

The components of surgical system 10 can be fabricated from biologically acceptable materials suitable for medical applications, including metals, synthetic polymers, ceramics and/or their composites, depending on the particular application and/or preference of a medical practitioner. For example, the components of surgical system 10, individually or collectively, can be fabricated from materials such as stainless steel alloys, aluminum, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, composites of PEEK and calcium based ceramics, and composites of PEEK with resorbable polymers. Various components of surgical system 10 may have material composites, including the above materials, to achieve various desired characteristics such as strength, rigidity, elasticity, compliance, biomechanical performance, durability and radiolucency or imaging preference. The components of surgical system 10, individually or collectively, may also be fabricated from a heterogeneous material such as a combination of two or more of the above-described materials. The components of surgical system 10 may be monolithically formed, integrally connected or include fastening elements and/or instruments, as described herein.

Surgical system 10, is employed, for example, with minimally invasive, mini-open and/or open procedures for supporting tissue and/or anatomical structures to expose tissue and/or anatomical structures to create a surgical pathway and provide access to a surgical site, which includes, for example, a spine to facilitate treatment.

Surgical system 10 includes a retractor assembly 12 having a base 14 configured for connection with a frame 16. In one embodiment, retractor assembly 12 includes base 14 without frame 16. It is envisioned that the base may be formed with, connected and/or attached to, or the frame may include, a mounting apparatus disposed in a fixed, movable or mobile and/or a detachable or removable configuration. It is further envisioned that the mounting apparatus may be disposed adjacent a surgical site and supported with a surface of a body, extend from a surgical table and/or other surgical instrumentation, and/or hand held. In one embodiment, the base and/or the frame may be connected with a detachable handle.

A blade 18 is connected to base 14. Blade 18 extends between a first end 20 and a second end 22 and defines a longitudinal axis L1. Blade 18 includes an outer surface 24 defining a first side 26, a second side 28, lateral side 27 and lateral side 29. It is contemplated that all or only a portion of outer surface 24 includes various surface configurations, such as, for example, smooth, rough, mesh, porous, semi-porous, dimpled and/or textured according to the requirements of a particular application. It is envisioned that all or only a portion of blade 18 may have alternate cross section configurations, such as, for example, cylindrical, flat, oval, oblong, triangular, square, polygonal, irregular, uniform, non-uniform, offset, staggered, undulating, arcuate, variable and/or tapered. The width of first side 26 and second side 28 is configured to trap tissue and can be varied for a particular procedure. In one embodiment, blade 18 has a thin and/or low profile configuration. It is contemplated that blade 18 prevents surface 24 from catching or snagging undesired anatomy and/or without unnecessarily disrupting tissue and/or anatomical structures.

Blade 18 includes a cavity, such as, for example, a plurality of recesses 30. Recesses 30 have an angled cutout configuration and are configured to facilitate articulating movement of second end 22 relative to blade 18. It is contemplated that recesses 30 are spaced apart and disposed along second side 28 at second end 22. Each recess 30 defines a first surface 32 and a second surface 34. Second surface 34 faces first surface 32. It is contemplated that first surface 32 and second surface 34 include various surface configurations, such as, for example, smooth, rough, mesh, porous, semi-porous, dimpled and/or textured according to the requirements of a particular application. In one embodiment, each recess 30 has an arcuate configuration disposed along blade 18. For example, the arcuate configuration includes a plurality of recesses disposed in a nested cup orientation to facilitate articulating movement of second end 22 relative to blade 18. It is envisioned that blade 18 may include one or a plurality of cavities. It is contemplated that the cavity may comprise various configurations, such as, for example, a notch, detent, opening or groove.

Blade 18 defines a longitudinal cavity 36. Longitudinal cavity 36 extends between first end 20 and second end 22 along axis L1. Longitudinal cavity 36 is configured for disposal of a longitudinal element, discussed below.

Blade 18 includes a first member, such as, for example, a longitudinal element 38. Longitudinal element 38 is disposed with first end 20 and connected to second end 22. Longitudinal element 38 is configured for disposal in longitudinal cavity 36. Longitudinal element 38 is connected to second end 22 by a connection mechanism 42, such as, for example, nut, monolithic enlarged part, tied knot, friction fit, pressure fit, locking protrusion/recess, locking keyway, welding and/or adhesive. In one embodiment, element 38 includes a cable that can be made of various materials, such as, for example, part solid rod and/or part braided cable, part solid rod and part Nitinol wire, all cable or all Nitinol wire. It is contemplated that longitudinal element 38 is monolithically connected with the surface that defines cavity 36. It is contemplated that longitudinal element 38 is a separate element being disposed in cavity 36. It is contemplated that longitudinal element 38 is moveably connected to cavity 36.

Longitudinal element 38 is actuated with an engagement mechanism, such as, for example, a collar 40 to dispose second end 22 between a first linear orientation (FIG. 1) axially extending along axis L1 and a second non-linear orientation (FIG. 2) transverse to axis L1. In the first orientation, surface 32 and surface 34 are spaced apart. As collar 40 is rotated, element 38 is tensioned such that a rod or cable of element 38 is pulled taught. Element 38 draws mechanism 42 such that second end 22 moves relative to blade 18 such that the tip of blade 18 is pulled into a curved configuration.

Blade 18 is articulated from the first orientation to the second orientation and each surface 32 and surface 34 converge. Surface 32 converges towards surface 34 closing the space between the surfaces to facilitate end 22 articulating into a non-linear configuration such that the tip of blade 18 articulates to form a curved configuration. This configuration allows blade 18 to be inserted having a low profile and formed to a selected configuration to retract tissue and/or anatomical structures. It is contemplated that the curved blade 18 holds back desired anatomical structures from a working channel at a surgical site.

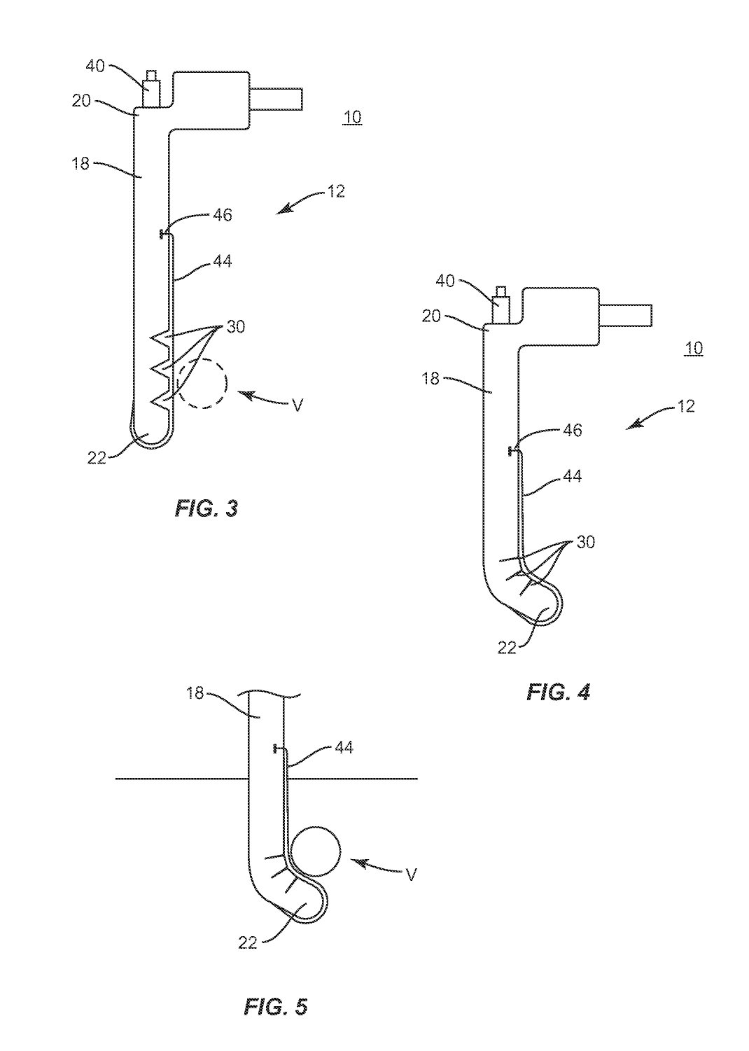

In one embodiment, as shown in FIGS. 3 and 4, retractor assembly 12, similar to that described with regard to FIGS. 1 and 2, includes a protective cover, such as, for example, a sheath 44. Sheath 44 is provided to cover recesses 30 and prevent recesses 30 from undesirably pinching, snagging, catching and/or damaging tissue and/or anatomical structures disposed adjacent blade 18 while surfaces 32, 34 converge and engage, as shown in FIG. 4. Sheath 44 is connected to blade 18 via a connecting mechanism, such as, for example, a recess 46. It is contemplated alternate connecting mechanisms can be utilized, such as, for example, friction fit, pressure fit, locking protrusion/recess, locking keyway and/or adhesive. In one embodiment, sheath 44 is disposed to cover only recesses 30. In one embodiment, sheath 44 encloses blade 18 completely. It is envisioned that sheath 44 may cover only a portion, substantially all and/or a circumferential portion of blade 18.

In assembly, operation and use, as shown in FIGS. 1-5, surgical system 10, similar to that described above, is employed, for example, with a minimally invasive surgical procedure for spinal and neurosurgical applications with a patient. For example, during spine surgery, a surgeon will make an incision in the skin of a patient's back over vertebrae to be treated. One or more dilators may be employed to gradually separate the muscles and create a portal through which the surgery may be performed.

Retractor assembly 12 is positioned adjacent the surgical site over the small incision. Retractor assembly 12 is passed through the incision to create a passageway or portal to the surgical site. Blade 18 is disposed at the surgical site in a linear orientation relative to axis L1, as shown in FIGS. 1 and 3. The surface of sheath 44, disposed about blade 18, engages tissue, such as, for example, a vessel V. It is contemplated that the surfaces of assembly 12 may engage tissue including soft tissue, ligaments, tendons, cartilage and/or bone.

Blade 18 is selectively articulated to space apart tissue and create access and/or a surgical pathway to a surgical site. Element 38 includes a cable that is actuated with collar 40 to dispose second end 22 between a linear orientation (FIGS. 1 and 3) axially extending along axis L1 and a non-linear orientation (FIGS. 2, 4 and 5) transverse to axis L1. Collar 40 is rotated, in the direction shown by arrow A in FIG. 2, and the cable of element 38 is tensioned. The cable of element 38 draws mechanism 42 such that end 22 moves relative to blade 18 such that the tip of blade 18 is pulled into a curved configuration, as shown by arrow B, to engage and space apart vessel V from other tissue at the surgical site.

Blade 18 is articulated from the first orientation to the second orientation and each surface 32 converges towards surface 34 closing the space between the surfaces to facilitate end 22 articulating into a non-linear configuration such that the tip of blade 18 articulates to form a curved configuration. Sheath 44 protects vessel V and/or other tissue and anatomical structures adjacent the surgical site from being damaged during the surgical procedure, as shown in FIG. 5. It is contemplated that the tip of blade 18 and/or portions of blade 18 may be disposed in one or a plurality of orientations between the first orientation and the second orientation. It is contemplated that the tip of blade 18 may assume alternative configurations relative to blade 18, such as, for example, perpendicular, linear, undulating, staggered, uniform, non-uniform and spherical.

Upon completion of the surgical procedure, collar 38 is rotated in the direction shown by arrow C to release tension from element 38. End 22 moves relative to blade 18, as shown by arrow D. Blade 18 is articulated from the second orientation to the first orientation and surface 32 is spaced apart from surface 34 to facilitate end 22 articulating into a linear configuration. Sheath 44 protects vessel V and/or other tissue and anatomical structures as blade 18 articulates to the linear orientation. Retractor assembly 12 is removed from the surgical site.

It is envisioned that the use of microsurgical and image guided technologies may be employed to access, view and repair spinal deterioration or damage, with the aid of retractor assembly 12. It is contemplated that a surgical procedure may employ other instruments that can be mounted with retractor assembly 12, such as, for example, nerve root retractors, tissue retractors, forceps, cutter, drills, scrapers, reamers, separators, rongeurs, taps, cauterization instruments, irrigation and/or aspiration instruments, illumination instruments and/or inserter instruments.

Retractor assembly 12 may be employed for performing spinal surgeries, such as, for example, laminectomy, discectomy, fusion, laminotomy, nerve root retraction, foramenotomy, facetectomy, decompression, spinal nucleus or disc replacement and procedures using bone graft and implantable prosthetics including plates, rods, and bone engaging fasteners.

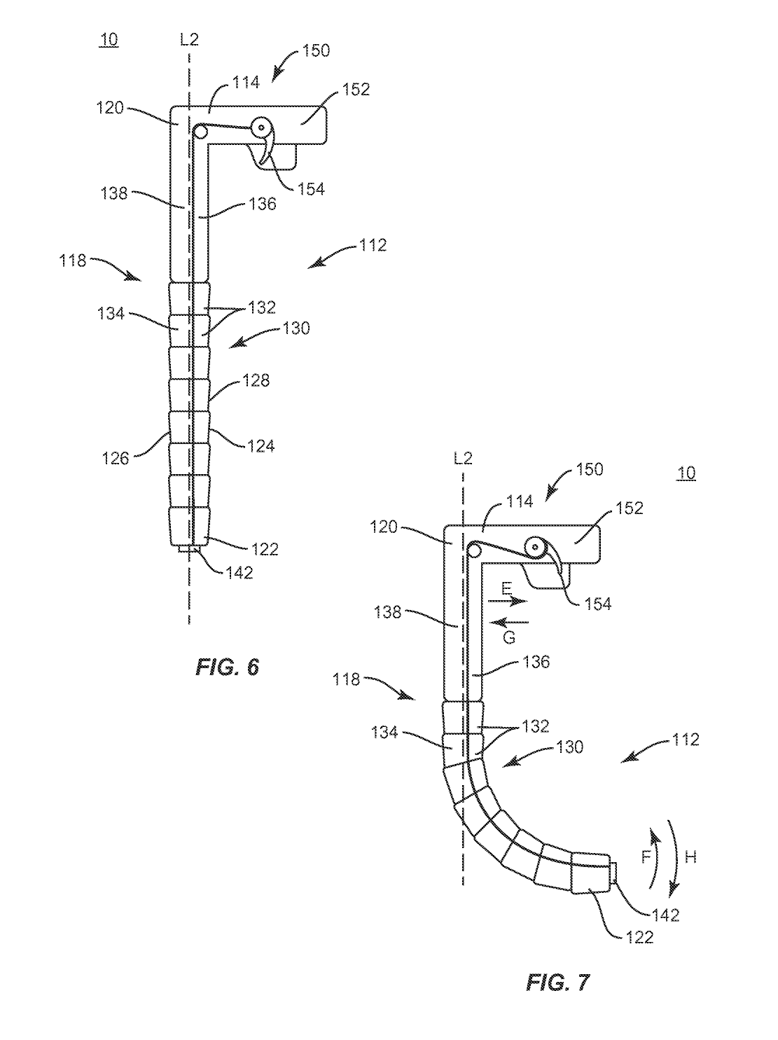

In one embodiment, as shown in FIGS. 6 and 7, surgical system 10, similar to the systems and methods described with regard to FIGS. 1-5, includes a retractor assembly 112 having a base 114 configured for connection with a frame (not shown), similar to that described herein. A blade 118 is connected to base 114. Blade 118 extends between a first end 120 and a second end 122 and defines a longitudinal axis L2. Blade 118 includes an outer surface 124 configured for engaging and spacing apart tissue, and defining a first side 126 and a second side 128.

Blade 118 includes a link configuration 130. Link configuration 130 includes a series of interconnected links 132, which are relatively movable such that link configuration 130 is disposable between a flexible configuration, to facilitate articulation and a relatively rigid configuration to facilitate disposal of blade 118 in a non-linear orientation, such as, for example, a curved configuration to engage and space apart tissue at a surgical site, similar to that described herein. Each link 132 has an outer surface 134. It is contemplated that all or only a portion of each outer surface 134 may have alternate surface configurations, such as, for example, rough, undulating, porous, semi-porous, dimpled, polished and/or textured according to the requirements of a particular application.

Blade 118 defines a longitudinal cavity 136 disposed through link configuration 130. Longitudinal cavity 136 extends between first end 120 and second end 122 along axis L2. Longitudinal cavity 136 is configured for disposal of a longitudinal element, discussed below.

Blade 118 includes a first member, such as, for example, a longitudinal element 138. Longitudinal element 138 is disposed with first end 120 and connected to second end 122. Longitudinal element 138 is configured for disposal in longitudinal cavity 136. Longitudinal element 138 is connected to second end 122 by a connection mechanism 142, such as, for example, nut, monolithic enlarged part, tied knot, friction fit, pressure fit, locking protrusion/recess, locking keyway, welding and/or adhesive.

Longitudinal element 138 is actuated with an engagement mechanism 150 to dispose second end 122 between a first linear orientation (FIG. 6) axially extending along axis L2 and a second non-linear orientation (FIG. 7) transverse to axis L2. Mechanism 150 includes a handle 152 and a trigger 154. Trigger 154 is disposed with first end 120 and connected to second end 122. In one embodiment, a locking mechanism (not shown) can be utilized to lock trigger 154 in place during the surgical procedure.

In the first orientation, link configuration 130 is disposed in a relatively flexible configuration such that links 132 are relatively movable. Trigger 154 is engaged and drawn in the direction shown by arrow E in FIG. 7 such that a rod or cable of element 138 is tensioned. The rod or cable of element 138 draws mechanism 142 such that links 132 are tensioned. End 122 moves relative to blade 118 such that the tip of blade 118 is articulated into a curved configuration, in the direction shown by arrow F. Link configuration 130 is drawn into a relatively rigid orientation to form the curved configuration of blade 118, similar to blade 18 described herein. This configuration allows blade 118 to be inserted having a low profile and formed to a selected configuration to retract tissue and/or anatomical structures. It is contemplated that the curved blade 118 holds back desired anatomical structures from a working channel at a surgical site.

Upon completion of the surgical procedure, trigger 154 is released in the direction shown by arrow G to release tension from element 138 and links 132. End 122 moves relative to blade 118, as shown by arrow H. Blade 118 is articulated from the second orientation to the first orientation such that link configuration 130 is disposed in a relatively flexible linear configuration. In one embodiment, blade 118 may include a protective cover, similar to sheath 44 described herein, to protect tissue and anatomical structures.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

1. A retractor comprising:
a base;
a blade connected to the base and extending between a first end including a threaded collar and a second end, the blade including at least one cavity configured to facilitate movement of the second end relative to the blade; and
a first member disposed with the threaded collar and connected to the second end, wherein the first member is engageable to dispose the second end between a first linear orientation and a second non-linear orientation.

2. A retractor as recited in claim 1, wherein the at least one cavity includes at least one recess.

3. A retractor as recited in claim 1, wherein the at least one cavity includes a plurality of spaced apart angled cutouts disposed along the second end.

4. A retractor as recited in claim 1, wherein the at least one cavity includes an arcuate configuration.

5. A retractor as recited in claim 1, wherein the blade defines a longitudinal cavity configured for disposal of the first member, which includes a longitudinal element that connects the first member to the second end.

6. A retractor as recited in claim 5, wherein the longitudinal element includes a cable.

7. A retractor as recited in claim 1, wherein the blade includes a first side and a second side configured to engage tissue, the second side including the at least one cavity.

8. A retractor as recited in claim 1, wherein the at least one cavity defines a first surface and a second surface facing the first surface such that in a first orientation the first surface and the second surface are spaced apart and in a second orientation the first surface and the second surface converge.

9. A retractor as recited in claim 1, further comprising a sheath disposed about the at least one cavity.

10. A retractor as recited in claim 1, wherein the first member is axially translatable to dispose the second end in the second non-linear orientation.

11. A retractor as recited in claim 8, wherein the first member includes a lock configured to alternatively fix the second end of the blade between the first orientation and the second orientation.

12. A retractor comprising:
a base configured for connection with a frame;
a blade connected to the base and extending between a first end including a threaded collar and a second end, the blade defining a longitudinal axis, the blade including an inner surface that defines a longitudinal cavity and at least one recess configured to facilitate movement of the second end relative to the blade; and
a longitudinal element disposed with the longitudinal cavity and the threaded collar and being connected to the second end, wherein the longitudinal element is engageable to dispose the second end between a first linear orientation axially extending along the longitudinal axis and a second non-linear orientation transverse to the longitudinal axis.

13. A retractor as recited in claim 12, wherein the at least one recess includes a plurality of spaced apart angled cutouts disposed along the second end.

14. A retractor as recited in claim 12, wherein the blade includes a first side and a second side configured to engage tissue, the second side including the at least one recess.

15. A retractor as recited in claim 12, wherein the at least one recess defines a first surface and a second surface facing the first surface such that in a first orientation the first surface and the second surface are spaced apart and in a second orientation the first surface and the second surface converge to engage.

16. A retractor as recited in claim 12, further comprising a sheath disposed about at least a second end of the blade.

17. A retractor as recited in claim 12, wherein the longitudinal element is axially translatable to dispose the second end in the non-linear orientation.

18. A retractor as recited in claim 15, wherein the longitudinal element includes a lock configured to alternatively fix the second end between the first orientation and the second orientation.

19. A retractor as recited in claim 2, wherein the at least one recess includes an arcuate configuration.

20. A retractor comprising:
a base configured for connection with a frame;
a blade connected to the base and extending between a first end including a threaded collar and a second end, the blade defining a longitudinal axis, the blade including an inner surface that defines a longitudinal cavity and an outer surface that defines a plurality of angled cutouts, the cutouts being disposed along the outer surface to define a first surface and a second surface facing the first surface;
a cable disposed with the threaded collar and connected to the second end; and
a sheath being disposed with the second end and movable therewith,
wherein the threaded collar is rotatable to tension the cable, which is engageable with the second end to dispose the second end between a first configuration such that the second end is disposed in a linear orientation extending along the longitudinal axis and a second configuration such that the second end is disposed in an arcuate orientation relative to the longitudinal axis.
